SEQUENCE LISTING (15 sequences)

SEQ ID NO: 1 - protein, 120 aa, Artificial Sequence
  Anti-CEA VHH
EVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSS

SEQ ID NO: 2 - protein, 102 aa, Artificial Sequence
  Anti-CD16 VHH
EVQLVESGGGLVQPGGSLRLSCSFPGSIFSLTMGWYRQAPGKERELVTSATPGGDTNYADFVKGRFTISRDNARSIIYLQMNSLKPEDTAVYYCYARTRNWG

SEQ ID NO: 3 - protein, 102 aa, Artificial Sequence
  Anti-CD16 VHH
EVQLVESGGELVQAGGSLRLSCAASGLTFSSYNMGWFRRAPGKEREFVASITWSGRDTFYADSVKGRFTISRDNAKNTVYLQMSSLKPEDTAVYYCAANPWP

SEQ ID NO: 4 - protein, 102 aa, Artificial Sequence
  Anti-CD16 VHH
EVQLVESGGGLVQPGESLTLSCVVAGSIFSFAMSWYRQAPGKERELVARIGSDDRVTYADSVKGRFTISRDNIKRTAGLQMNSLKPEDTAVYYCNAQTDLRD

SEQ ID NO: 5 - protein, 102 aa, Artificial Sequence
  Anti-CD16 VHH
EVQLVESGGGLVQPGGSLTLSCVAAGSIFTFAMSWYRQAPRKERELVARIGTDDETMYKDSVKGRFTISRDNVKRTAGLQMNNLKPEDTAVYYCNARTDYRD

SEQ ID NO: 6 - protein, 123 aa, Artificial Sequence
  Anti-Her2 sdAb
QVQLVQSGGGLVQAGGSLRLSCAASGRTFSSYAMAWFRQAPGKEREFVAAISWSGANIYVADSVKGRFTISRDNAKDTVYLQMNSLKPEDTAVYYCAVKLGFAPVEERQYDYWGQGTQVTVSS

SEQ ID NO: 7 - protein, 128 aa, Artificial Sequence
  Anti-EGFR1 VHH
MAEVQQASGGGLVQAGGSLRLSCAASGRTETTSAIAWFRQAPGKEREFVAQISASGLGINYSGTVKGRFTISRDADKTTVYLQMNSLTPEDTAVYYCAAGFHYIAAIRRTTDFHFWGPGTLVTVSSGR

SEQ ID NO: 8 - protein, 121 aa, Artificial Sequence
  Anti-F4+ETEC bacteria
QVQLQESGGGLVQAGGSLRLSCEASGNVDRIDAMGWFRQAPGKQREFVGYISEGGILNYGDFVKGRFTISRDNAKNTVYLQMSNLKSEDTGVYFCAASHWGTLLIKGIEHWGKGTQVTVSS

SEQ ID NO: 9 - protein, 127 aa, Artificial Sequence
  Anti-PS2-8 VHH
EVQLVESGGGLVQAGGSLRLSCAASGRSFSRDAMGWFRQAPGKERDVVAAINLNGGRTYSADSVKGRFTISRDNDKNTVYLQMSNLKPEDTAVYYCAAREGDVGLVSYKRSSNYPYWGQGTQVTVSS

SEQ ID NO: 10 - protein, 121 aa, Artificial Sequence
  Anti-Huntavirus VHH
MAEVQLQASGGGLVQAGGSLRLSCAASGRTSSMYSMVWFRQAPGKEREFVAGIIWTSSLTYYADSLKGRFTISRDNAKNTVYLQMNSLKPEDTAIYYCAADTKTGGGGYEYWGQVTVTVSS

SEQ ID NO: 11 - protein, 119 aa, Artificial Sequence
  Anti-Huntavirus VHH
MAEVQLQASGGGLVQPGGSLRLSCAASGSIFSSDVMGWFRQAPGKERELVAFITDDGGTNYADSVKGRFTISRDNAENTVSLQMNSLKPEDTAVYYCNARYYSGGYRNYWGQVTVTVSS

SEQ ID NO: 12 - protein, 115 aa, Artificial Sequence
  Anti-CD16 VHH
EVQLVESGGGLVQPGGSLRLSCSFPGSIFSLTMGWYRQAPGKERELVTSATPGGDTNYADFVKGRFTISRDNARSIIYLQMNSLKPEDTAVYYCYARTRNWGTVWGQGTQVTVSS

SEQ ID NO: 13 - protein, 129 aa, Artificial Sequence
  Anti-CD3 VHH
QVQLQESGGGLVQAGGSLRLSCAASGRTFSNYHMGWFRQAPGKERELVAAISGSGGSTYYTDSVKGRFTISRNNAKNTMSLQMSNLKPEDTGVYYCTTPTEKGSSIDYWGQGTQVTVSSGRYPYDVPDY

SEQ ID NO: 14 - protein, 227 aa, Artificial Sequence [sequence omitted]

SEQ ID NO: 15 - protein, 227 aa, Artificial Sequence
  Synthetic
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

(19) United States
(12) Patent Application Publication
(10) Pub. No.: US 2016/0280795 A1
(43) Pub. Date: Sep. 29, 2016

(54) BISPECIFIC ANTIBODY WITH TWO SINGLE-DOMAIN ANTIGEN-BINDING FRAGMENTS

(71) Applicant: Zhong Wang, Foster City, CA (US)

(72) Inventor: Zhong Wang, Foster City, CA (US)

(21) Appl. No.: 15/178,169

(22) Filed: Jun. 9, 2016

Related U.S. Application Data
(63) Continuation of application No. PCT/US2014/070985, filed on Dec. 17, 2014.
(60) Provisional application No. 61/918,383, filed on Dec. 19, 2013.

(51) Int. Cl. C07K 16/30; C07K 16/40; C07K 16/28

(52) U.S. Cl. CPC C07K 16/3007; C07K 16/283; C07K 16/2809; C07K 16/40; C07K 2317/35; C07K 2317/31; C07K 2317/524; C07K 2317/526; C07K 2317/569; C07K 2317/76

(57) ABSTRACT

Provided are bivalent bispecific antibody comprising a first polypeptide comprising a first Fc fragment and a first single-domain antigen-binding (VHH) fragment and a second polypeptide comprising a second Fc fragment and a second single-domain antigen-binding (VHH) fragment, wherein the first VHH fragment has specificity to a tumor cell or a microorganism and the second VHH fragment has specificity to an immune cell, and wherein the first fragment is N-terminal to the second fragment.

### CROSS REFERENCE TO RELATED APPLICATIONS

This application is a continuation of International Application No. PCT/US2014/070985, filed Dec. 17, 2014, which claims the benefit under 35 U.S.C. §119(e) of U.S. Provisional Application No. 61/918,383 filed on Dec. 19, 2013, which is hereby incorporated by reference in its entirety.

### BACKGROUND

Bispecific antibodies (BsMAb, BsAb) are artificial proteins composed of fragments of two different monoclonal antibodies and consequently binds to two different types of antigens. In cancer immunotherapy, for instance, BsMAbs are engineered that simultaneously bind to a cytotoxic cell and a target like a tumor cell to be destroyed.

At least three types of bispecific antibodies have been proposed or tested, including trifunctional antibody, chemically linked Fab and bi-specific T-cell engager. In order to overcome manufacturing difficulties, a first-generation BsMAb, called trifunctional antibody, has been developed. It consists of two heavy and two light chains, one each from two different antibodies. The two Fab regions are directed against two antigens. The Fc region is made up from the two heavy chains and forms the third binding site; hence the name.

Other types of bispecific antibodies have been designed to overcome certain problems, such as short half-life, immunogenicity and side-effects caused by cytokine liberation. They include chemically linked Fabs, consisting only of the Fab regions, and various types of bivalent and trivalent single-chain variable fragments (scFvs), fusion proteins mimicking the variable domains of two antibodies. The furthest developed of these newer formats are the bi-specific T-cell engagers (BiTEs) and trifunctional antibodies.

Despite these advancements, there are still major challenges with bispecific antibodies, such as improving manufacturing efficiency, retaining immunogenicity and maintaining half-life.

### SUMMARY

The present disclosure provides a bispecific antibody that includes immunoglobulin Fc fragments connected to two single-domain antigen-binding fragments (or domains), each of which targets a different antigen. Such a bispecific antibody, without light chains and with its reduced molecule weight as compared to a conventional antibody, presents a significant advantage in antibody production and purification. Unexpectedly, such a bispecific antibody can still bind to both antigens effectively, carrying out its intended biological functions. Also unexpectedly, even though these disclosed bispecific antibodies are still heterodimmers, they can be readily expressed in bacterial cells such as E. coli, leading to production of soluble proteins, while other known bispecific antibodies produced by E. coli are hardly soluble.

Thus, one embodiment of the present disclosure provides a bivalent bispecific antibody comprising (a) a first polypeptide comprising a first Fc fragment and a first single-domain antigen-binding (VHH) fragment and (b) a second polypeptide comprising a second Fc fragment and a second single-domain antigen-binding (VHH) fragment, wherein the first VHH fragment has specificity to a tumor cell or a microorganism and the second VHH fragment has specificity to an immune cell.

In some aspects, the first VHH fragment has specificity to a tumor antigen. In some aspects, the tumor antigen is selected from the group consisting of CEA, EGFR, Her2, EpCAM, CD20, CD30, CD33, CD47, CD52, CD133, CEA, gpA33, Mucins, TAG-72, CIX, PSMA, folate-binding protein, GD2, GD3, GM2, VEGF, VEGFR, Integrin, αVβ3, α5β1, ERBB2, ERBB3, MET, IGFIR, EPHA3, TRAILR1, TRAILR2, RANKL, FAP and Tenascin. In some aspects, the tumor antigen is CEA.

In some aspects, the first VHH fragment comprises the amino acid sequence of SEQ ID NO:1, or an amino acid having at least about 95% sequence identity thereto.

In some aspects, the first VHH fragment has specificity to a virus or a bacterium. In some aspects, the first VHH fragment has specificity to an endotoxin.

In some aspects, the second VHH fragment has specificity to an antigen selected from the group consisting of CD3, CD16, CD19, CD28 and CD64. In some aspects, the antigen is CD16.

In some aspects, the second VHH fragment comprises the amino acid sequence of one of SEQ ID NO:2-5, or an amino acid having at least about 95% sequence identity thereto.

In some aspects, the first VHH fragment and/or the second VHH fragment does not contain Val, Gly, Leu, and Trp residues at Kabat positions 37, 44, 45, and 47, respectively.

In some aspects, each of the Fc fragments comprises a CH2 domain and a CH3 domain.

In some aspects, the two polypeptides are connected with two disulfide bonds. In some aspects, the disulfide bonds are connected between cysteine residues located at a hinge region between each of the VHH fragment and the Fc fragment.

In some aspects, the Fc fragments comprise one or more substitutions, as compared to a wild-type Fc fragment, that form an ionic bond between the Fc fragments.

In some aspects, the Fc fragments comprises one or more substitutions, as compared to a wild-type Fc fragments, that form a knob-into-the-hole conformational pairing between the heavy chain and the Fc fragment.

Also provided, in one embodiment, is a polynucleotide comprising a nucleic acid sequence encoding an antibody of any preceding claim. In some aspects, provided is a host cell comprising the polynucleotide of the present disclosure. In some aspects, the host cell is a bacterial cell or yeast cell. In some aspects, the host cell is E. coli.

Yet in another embodiment, provided is a method of treating a tumor in a patient, comprising administering to the patient an antibody of present disclosure, wherein the first fragment has specificity to a tumor antigen expressed on a tumor cell in the patient.

Another embodiment provides a polypeptide comprising the amino acid sequence of SEQ ID NO:13 or having at least 95% sequence identity to SEQ ID NO:13 (or having one, two or three amino acid addition, deletion or substitution as compared to SEQ ID NO:13), wherein the polypeptide has binding specificity to a mammalian CD3 protein, such as a human CD3 protein.

In some embodiments, the present disclosure provides a bivalent antibody comprising (a) a first polypeptide comprising a first Fc fragment and a first single-domain antigen-binding (VHH) fragment and (b) a second polypeptide comprising a second Fc fragment and a second VHH fragment. The two polypeptides can be identical (bivalent, monospecific antibody) or have different binding specificity (bivalent, bispecific antibody).

### BRIEF DESCRIPTION OF THE DRAWINGS

FIG. 1 illustrates a bispecific antibody that includes a first single-domain antigen-binding fragment (VHH1) and a second VHH (VHH2) each connected to the N-terminus of an Fc fragment, forming a light chain-less bispecific antibody.

FIG. 2 presents a multiple sequence alignment of the sequences of Table 1, with related domains annotated.

FIG. 3 shows Commassiue blue staining of insoluble and soluble fractions of the antibody expressed in the bacterial cells.

FIG. 4 shows the staining of each of the two antibody chains separately, using antibodies against the His tag and the Flag tag, respectively.

FIG. 5 shows Western blots with anti-His tag antibody (middle panel) or with anti-Flag tag antibody (lower panel), as compared to Commassiue blue staining of the purified antibodies (upper panel).

FIG. 6-8 present Commassiue blue staining images for obtained bispecific antibodies at each stage of purification, for the anti-CEA-Fc:anti-CD3-Fc, the anti-Her2-Fc:anti-CD16-Fc and the anti-Her2-Fc:anti-CD3-Fc bispecific antibodies, respectively.

FIG. 9-11 present bar charts showing the cytotoxicity of three bispecific antibodies, anti-CEA-Fc:anti-CD16-Fc (FIG. 9) anti-CEA-Fc:anti-CD3-Fc (FIG. 10) anti-CEA-Fc:anti-CD16-Fc (FIG. 11) in a killing manner dependent on bispecific antibodies.

FIG. 12 presents a bar chart showing the dose-dependent and immune cell-dependent nature of the cytotoxicity in a killing manner dependent on bispecific antibody.

### DETAILED DESCRIPTION

### Definitions

It is to be noted that the term “a” or “an” entity refers to one or more of that entity; for example, “a bispecific antibody,” is understood to represent one or more bispecific antibodies. As such, the terms “a” (or “an”), “one or more,” and “at least one” can be used interchangeably herein.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art.

The term “an equivalent polynucleotide” refers to a nucleic acid sequence having a certain degree of homology, or sequence identity, to a reference nucleotide sequence or the complement thereof. In one aspect, homologs of nucleic acids are capable of hybridizing to the nucleic acid or complement thereof. Likewise, “an equivalent polypeptide” refers to a polypeptide having a certain degree of homology, or sequence identity, with the amino acid sequence of a reference polypeptide. In some aspects, the sequence identity is at least about 70%, 75%, 80%, 85%, 90%, 95%, 98%, or 99%. In some aspects, the equivalent sequence retains the activity (e.g., epitope-binding) or structure (e.g., salt-bridge) of the reference sequence.

For each polypeptide or polynucleotide disclosed herein, its equivalents are also contemplated. In one aspect, an equivalent of a polypeptide includes an alteration (i.e., a deletion, an addition or a substitution) of an amino acid residue. In one aspect, an equivalent of a polypeptide includes no more than two alterations of amino acid residues. In one aspect, an equivalent of a polypeptide includes no more than 3, 4, or 5 alterations of amino acid residues. In some aspects, the amino acid alterations are at residues not critical to the activity of the reference polypeptide. Residues critical to the activity of a polypeptide can be readily tested by site-specific mutation analysis, or even sequence alignments as such residues are highly reserved.

As used herein, an “antibody” or “antigen-binding polypeptide” refers to a polypeptide or a polypeptide complex that specifically recognizes and binds to one or more antigens. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. The term antibody also encompasses polypeptides or polypeptide complexes that, upon activation, possess antigen-binding capabilities.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgGI, IgG2, IgG3, IgG4, IgA1 and IgA2) or subclass of immunoglobulin molecule.

Light chains are classified as either kappa or lambda (K, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VK) and heavy (VH) chain portions determine antigen recognition and specificity. Conversely, the constant domains of the light chain (CK) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminal portion is a variable region and at the C-terminal portion is a constant region; the CH3 and CK domains actually comprise the carboxy-terminus of the heavy and light chain, respectively.

As indicated above, the variable region allows the antibody to selectively recognize and specifically bind epitopes on antigens. That is, the VK domain and VH domain, or subset of the complementarity determining regions (CDRs), of an antibody combine to form the variable region that defines a three dimensional antigen-binding site. This quaternary antibody structure forms the antigen-binding site present at the end of each arm of the Y. More specifically, the antigen-binding site is defined by three CDRs on each of the VH and VK chains (i.e. CDR-H1, CDR-H2, CDR-H3, CDR-L1, CDR-L2 and CDR-L3). In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

In naturally occurring antibodies, the six “complementarity determining regions” or “CDRs” present in each antigen-binding domain are short, non-contiguous sequences of amino acids that are specifically positioned to form the antigen-binding domain as the antibody assumes its three dimensional configuration in an aqueous environment. The remainder of the amino acids in the antigen-binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a particular CDR will vary depending on the sequence and size of the CDR. Those skilled in the art can routinely determine which residues comprise a particular CDR given the variable region amino acid sequence of the antibody.

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D”.

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

### Bivalent Antibodies

The present disclosure, in one embodiment, provides bispecific antibodies targeting two different antigens, one of which is present on a tumor cell or a microorganism, and another on an immune cell. Upon administration to an individual, the bispecific antibody specifically binds to a tumor cell or microorganism and at the same time specifically binds immune cells, such as a cytotoxic cell. Such dual binding can lead to killing of the bound tumor or microorganism by the host's immune system.

The bispecific antibody of the present technology includes two single-domain antigen-binding fragments (VHH fragments or VHH domains), each having specificity to one of the antigens. VHH fragments are known in the art and are further described below. Each of the VHH fragments is connected, optionally through a hinge region, to an Fc fragment of a conventional antibody.

As a VHH fragment is independently capable of specifically recognizing and binding an antigen without a paired light chain, such an antibody includes only two polypeptide chains. Accordingly, during production of the bispecific antibody, there are only two possible pairings for each chain. In other words, even without any paring selection, about half (50%) of paired antibodies would be the desired bispecific antibody. Given the lack of light chains alone, therefore, the present technology leads to greatly improved production efficiency compared to conventional Fab-Fc bispecific antibodies.

### A. Unexpected Properties of the Presently Disclosed Bispecific Antibodies

As a VHH fragment is smaller and much shorter than conventional Fab fragments, it was suspected that a bispecific antibody that includes two VHH fragments could be too short to render functional dual specificity. This is because a bispecific antibody, such as one that simultaneously targets two cells, an immune cell and a tumor cell or bacterium, needs access to specific antigens on two separate large cells. Too short a connection between the two antigen-binding sites on a bispecific antibody, therefore, would subject the antibody to steric hindrance between the two cells.

Unexpectedly, however, it is discovered that the suspected steric hindrance had limited impact on the function of the presently disclosed bispecific antibodies. Further, the overall bispecific affinity of the antibody, without the need of light chains, was comparable to conventional bispecific antibodies (see Example 3).

The advantage of the present technology also applies to cell expression efficiency and protein stability. Even though it was generally thought that small antibodies are easier to produce in cells than larger antibodies, bispecific antibodies present a special challenge for antibody production. This is at least because the two (or three or four) different protein chains can interact with one another inside and outside cells, leading to interference and thus decreased efficiency of a host cell's expression system and increased protein instability. Therefore, bispecific antibodies with full heavy chains and light chains are typically expressed in two separate cells, or use a common light chain to reduce mis-pairing of light chains with heavy chains, which produce non-functional antibodies.

Even bispecific antibodies that are smaller than those presently disclosed are associated with such problems. For instance, Bi-specific T-cell engagers (BITEs) are fusion proteins consisting of two typical single chain variable fragments. Although much smaller (typically 55 KDa) than the bispecific antibodies disclosed herein (about 100 KDa), BITEs cannot be expressed soluble in bacterial cells. Presently, BITEs are expressed in mammalian systems, which are much more expensive.

Unexpectedly, as demonstrated in Examples 1-2, when expressed in E. coli cells, up to about 20% the bispecific antibodies of the present disclosure were soluble. As explained above, these antibodies are much larger than BITEs. Nevertheless, while bacterium-expressed BITEs are entirely insoluble, the bispecific antibodies of the present disclosure can be readily produced from bacterial cells. Such a result, therefore, is surprising and unexpected.

Furthermore, since the bispecific antibodies of the present technology have relatively small sizes compared to conventional antibodies, and they are efficient to produce in particular in a large-scale setting, such as from yeast and bacterial hosts. The stability, solubility and half-life of these bispecific ligands are also much superior to the bispecific antibodies being developed in the field.

### B. Bivalent, Monospecific or Bispecific Antibodies

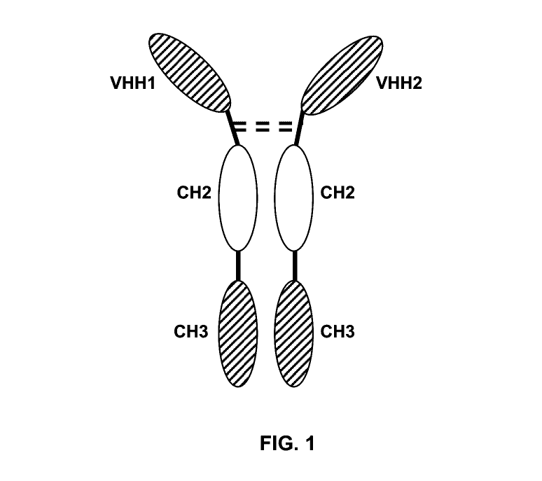

From the foregoing, it is apparent that the antibodies of the structures as illustrated in FIG. 1, in general, exhibit high bacterial production, stability and binding affinity. In some embodiments, therefore, the present disclosure provides bivalent antibody comprising (a) a first polypeptide comprising a first Fc fragment and a first single-domain antigen-binding (VHH) fragment and (b) a second polypeptide comprising a second Fc fragment and a second VHH fragment. Such a bivalent antibody can be monospecific (when the two polypeptides are identical or have the same binding specificity) or bispecific (when the two polypeptides have different binding specificity).

Such a bispecific antibody can be configured to target different antigen pair. For instance, one VHH fragment can have specificity to a first immune cell while the other target a second immune cell; one VHH fragment can have specificity to a first tumor cell while the other has specificity to a second tumor cell; one VHH fragment can have specificity to an immune cell and the other to a microorganism, an infected cell, a tumor cell, a inflamed cell, an apoptotic cell, or a foreign cell, without limitation.

### C. Single-Domain Antigen-Binding Fragments (VHH)

A “single-domain antigen-binding fragment,” or “single-domain antibody fragment” or “VHH”, is an antigen-binding fragment that is able to bind to an antigen without pairing with a light chain. VHH was originally isolated from single-domain antibodies (sdAb) as the sole antigen-binding fragment. The first known single-domain antibodies were isolated from camelids (Hamers-Casterman et al., Nature 363:446-8 (1993) and later from cartilaginous fish. Camelids produce functional antibodies without light chains and their single N-terminal domain (VHH) binds antigen without requiring domain pairing (reviewed in Harmsen and Haard, App Microbiol Biotechnol., 77:13-22 (2007)). Single-domain antibodies do not include CH1 domains which, in a conventional antibody, interact with the light chains.

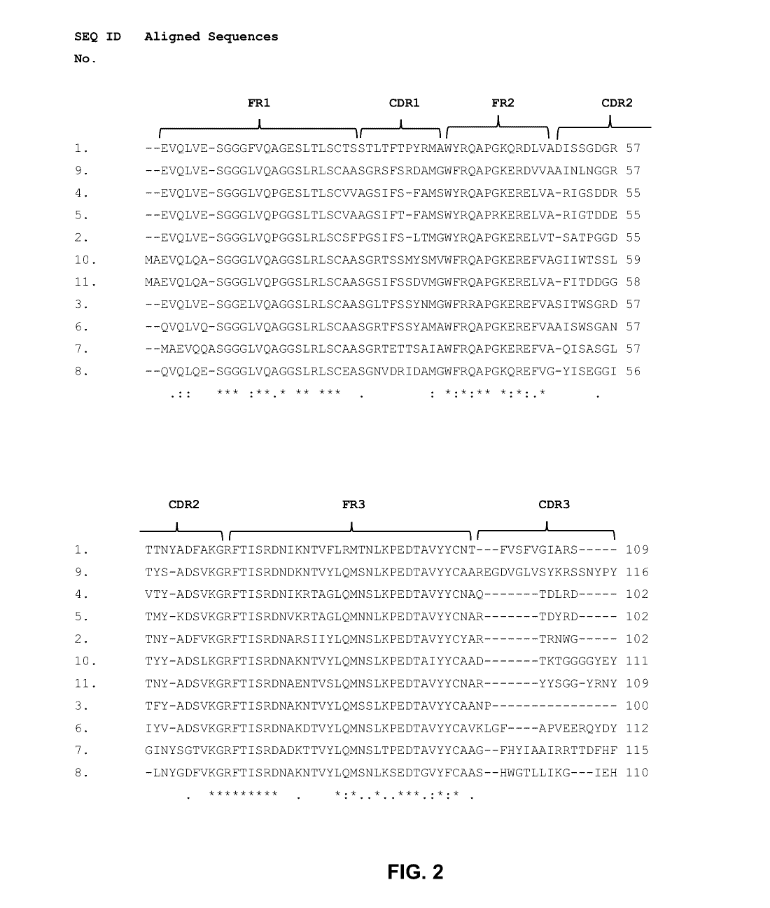

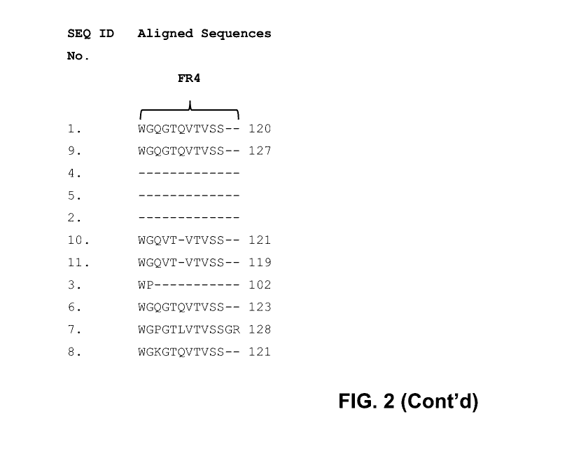

VHHs contain four framework regions (FR1-FR4) that form the core structure of the immunoglobulin domain and three complementarity-determining regions (CDR1-CDR3) that are involved in antigen binding (see, e.g., FIG. 2). As compared to human VH domains, The VHH framework regions show a high sequence homology (>80%) to human VH domains. See Harmsen and Haard, 2007, which further describes that the “most characteristic feature of VHHs is the presence of amino acid substitutions at four FR2 positions (positions 37, 44, 45, and 47; Kabat numbering) that are conserved in conventional VH domains and that are involved in forming the hydrophobic interface with VL domains.” VHHs typically have different amino acid residents at these and other positions that are highly reserved in the conventional VHs (e.g., Leu11Ser, Va137Phe or Tyr, Gly44Glu, Leu45Arg or Cys, Trp47Gly).

Also as described in Harmsen and Haard, 2007, CDRs of VHHs have certain known characteristic features. The N-terminal part of CDR1, for instance, is more variable and a conventional antibody. Further, some VHHs have an extended CDR3 that is often stabilized by an additional disulfide bond with a cysteine in CDR1 or FR2, resulting in the folding of the CDR3 loop across the former VL interface. A particular subfamily of llama VHHs (VHH3) also contains an extended CDR3 that is stabilized by an additional disulfide bond with a cysteine at position 50 in FR2.

Many sdAbs are known in the art, and can be readily prepared from animals such as camelids. From these sdAb, their VHHs can be readily identified and prepared. Table 1 lists a number of non-limiting examples for VHHs and sdAbs. Accordingly, in some embodiments, the present disclosure provides polypeptides comprising each of such disclosed sequences, the equivalents thereof, and polynucleotides encoding each. In one aspect, the polypeptide comprises an amino acid sequence of SEQ ID NO:13, or an amino acid sequence having one, two or three amino acid addition/deletion/substitution.

TABLE 1
Example Single-Domain Antigen-Binding Fragments (VHHs) and Single-
Domain Antibodies (sdAbs)

1. Anti-CEA VHH (SEQ ID NO: 1)
EVQLVESGGG FVQAGESLTL SCTSSTLTFT PYRMAWYRQA PGKQRDLVAD ISSGDGRTTN
YADFAKGRFT ISRDNIKNTV FLRMTNLKPE DTAVYYCNTF VSFVGIARSW GQGTQVTVSS
2. Anti-CD16 VHH (SEQ ID NO: 2)
EVQLVESGGG LVQPGGSLRL SCSFPGSIFS LTMGWYRQAP GKERELVTSA TPGGDTNYAD
FVKGRFTISR DNARSIIYLQ MNSLKPEDTA VYYCYARTRN WG
3. Anti-CD16 VHH (SEQ ID NO: 3)
EVQLVESGGE LVQAGGSLRL SCAASGLTFS SYNMGWFRRA PGKEREFVAS ITWSGRDTFY
ADSVKGRFTI SRDNAKNTVY LQMSSLKPED TAVYYCAANP WP
4. Anti-CD16 VHH (SEQ ID NO: 4)
EVQLVESGGG LVQPGESLTL SCVVAGSIFS FAMSWYRQAP GKERELVARI GSDDRVTYAD
SVKGRFTISR DNIKRTAGLQ MNSLKPEDTA VYYCNAQTDL RD
5. Anti-CD16 VHH (SEQ ID NO: 5)
EVQLVESGGG LVQPGGSLTL SCVAAGSIFT FAMSWYRQAP RKERELVARI GTDDETMYKD
SVKGRFTISR DNVKRTAGLQ MNNLKPEDTA VYYCNARTDY RD
6. Anti-Her2 sdAb (SEQ ID NO: 6)
QVQLVQSGGG LVQAGGSLRL SCAASGRTFS SYAMAWFRQA PGKEREFVAA ISWSGANIYV
ADSVKGRFTI SRDNAKDTVY LQMNSLKPED TAVYYCAVKL GFAPVEERQY DYWGQGTQVT
VSS
7. Anti-EGFR1 VHH (SEQ ID NO: 7)
MAEVQQASGG GLVQAGGSLR LSCAASGRTE TTSAIAWFRQ APGKEREFVA QISASGLGIN
YSGTVKGRFT ISRDADKTTV YLQMNSLTPE DTAVYYCAAG FHYIAAIRRT TDFHFWGPGT
LVTVSSGR
8. Anti-F4 + ETEC bacteria VHH (SEQ ID NO: 8)
QVQLQESGGG LVQAGGSLRL SCEASGNVDR IDAMGWFRQA PGKQREFVGY ISEGGILNYG
DFVKGRFTIS RDNAKNTVYL QMSNLKSEDT GVYFCAASHW GTLLIKGIEH WGKGTQVTVS
S
9. Anti-PS2-8 VHH (SEQ ID NO: 9)
EVQLVESGGG LVQAGGSLRL SCAASGRSFS RDAMGWFRQA PGKERDVVAA INLNGGRTYS
ADSVKGRFTI SRDNDKNTVY LQMSNLKPED TAVYYCAARE GDVGLVSYKR SSNYPYWGQG
TQVTVSS
10. Anti-Huntavirus VHH (SEQ ID NO: 10)
MAEVQLQASG GGLVQAGGSL RLSCAASGRT SSMYSMVWFR QAPGKEREFV AGIIWTSSLT
YYADSLKGRF TISRDNAKNT VYLQMNSLKP EDTAIYYCAA DTKTGGGGYE YWGQVTVTVS
S
11. Anti-Huntavirus VHH (SEQ ID NO: 11)
MAEVQLQASG GGLVQPGGSL RLSCAASGSI FSSDVMGWFR QAPGKERELV AFITDDGGTN
YADSVKGRFT ISRDNAENTV SLQMNSLKPE DTAVYYCNAR YYSGGYRNYW GQVTVTVSS
12. Anti-CD16 VHH (SEQ ID NO: 12)
EVQLVESGGG LVQPGGSLRL SCSFPGSIFS LTMGWYRQAP GKERELVTSA TPGGDTNYAD
FVKGRFTISR DNARSIIYLQ MNSLKPEDTA VYYCYARTRN WGTVWGQGTQVTVSS
13. Anti-CD3 VHH (SEQ ID NO: 13)
QVQLQESGGG LVQAGGSLRL SCAASGRTFS NYHMGWFRQA PGKERELVAA ISGSGGSTYY
TDSVKGRFTI SRNNAKNTMS LQMSNLKPED TGVYYCTTPT EKGSSIDYWG QGTQVTVSSG
RYPYDVPDY

As shown in FIG. 2, certain regions of these sequences are highly conserved, such as FR1-3, while the CDRs are more variable.

### D. Fc Modifications to Enhance Heterodimer Paring

The VHH fragments each is connected, optionally through a hinge region or linker, to the N-terminus of an Fc fragment, which is preferably a human Fc fragment or humanized Fc fragment. Modifications to the Fc fragments can be introduced to improve paring between two different antibody chains to form the desired bispecific antibody, or to further stabilize or improve activity of the antibodies. For instance, either or both of the Fc fragments can include one or more substitutions, as compared to a wild-type antibody Fc fragment, that form an ionic bond between them.

In one aspect, one of the Fc fragments contains one or more substitutions with amino acid residues having a positive charge under physiological conditions and the other Fc fragment contains one or more substitutions with one or more amino acid residues having a negative charge under physiological conditions. In one aspect, the positively charged amino acid residue can be arginine (R), histidine (H) or lysine (K). In another aspect, the negatively charged amino acid residue can be aspartic acid (D) or glutamic acid (E). Amino acid residues that can be substituted include, without limitation, D356, E357, L368, K370, K392, D399 and K409.

In some aspects, the Fc fragments can include one or more substitutions, as compared to a wild-type antibody Fc fragment, that form a knob-into-the-hole conformational pairing between them. Knob-into-hole designs are known in the art. See, e.g., Ridgway et al. “‘Knob-into-holes’ engineering of antibody C_{H}3 domains for heavy chain heterodimerization,” Protein Engineering 9(7):617-21 (1996).

In one aspect, K366 on one of the Fc fragment is substituted with a relatively large amino acid residue, such as tyrosine (Y) or tryptophan (W). Then Y407 on the other Fc fragment can be substituted with a relatively small amino acid residue, such as threonine (T), alanine (A) or valine (V).

### E. Binding Targets

In some embodiments, the first VHH (e.g., VHH1 of FIG. 1) of the bispecific antibody has binding specificity to a tumor antigen.

A “tumor antigen” is an antigenic substance produced in tumor cells, i.e., it triggers an immune response in the host. Tumor antigens are useful in identifying tumor cells and are potential candidates for use in cancer therapy. Normal proteins in the body are not antigenic. Certain proteins, however, are produced or overexpressed during tumorigenesis and thus appear “foreign” to the body. This may include normal proteins that are well sequestered from the immune system, proteins that are normally produced in extremely small quantities, proteins that are normally produced only in certain stages of development, or proteins whose structure is modified due to mutation.

An abundance of tumor antigens are known in the art and new tumor antigens can be readily identified by screening. Non-limiting examples of tumor antigens include EGFR, Her2, EpCAM, CD20, CD30, CD33, CD47, CD52, CD133, CEA, gpA33, Mucins, TAG-72, CIX, PSMA, folate-binding protein, GD2, GD3, GM2, VEGF, VEGFR, Integrin, αVβ3, a5β1, ERBB2, ERBB3, MET, IGF1R, EPHA3, TRAILR1, TRAILR2, RANKL, FAP and Tenascin.

In some aspects, the first VHH has specificity to a protein that is overexpressed on a tumor cell as compared to a corresponding non-tumor cell. A “corresponding non-tumor cell” as used here, refers to a non-tumor cell that is of the same cell type as the origin of the tumor cell. It is noted that such proteins are not necessarily different from tumor antigens. Non-limiting examples include carcinoembryonic antigen (CEA), which is overexpressed in most colon, rectum, breast, lung, pancreas and gastrointestinal tract carcinomas; heregulin receptors (HER-2, neu or c-erbB-2), which is frequently overexpressed in breast, ovarian, colon, lung, prostate and cervical cancers; epidermal growth factor receptor (EGFR), which is highly expressed in a range of solid tumors including those of the breast, head and neck, non-small cell lung and prostate; asialoglycoprotein receptor; transferrin receptor; serpin enzyme complex receptor, which is expressed on hepatocytes; fibroblast growth factor receptor (FGFR), which is overexpressed on pancreatic ductal adenocarcinoma cells; vascular endothelial growth factor receptor (VEGFR), for anti-angiogenesis gene therapy; folate receptor, which is selectively overexpressed in 90% of nonmucinous ovarian carcinomas; cell surface glycocalyx; carbohydrate receptors; and polymeric immunoglobulin receptor, which is useful for gene delivery to respiratory epithelial cells and attractive for treatment of lung diseases such as Cystic Fibrosis.

In one aspect, the first VHH has specificity to CEA or Her2. A representative sequence for this VHH is provided as SEQ ID NO:1 or 6 (Table 1). In some aspects, the first VHH includes an amino acid sequence of SEQ ID NO:1 or 6 with one or two or three addition, deletion or substitution. In one aspect, the first VHH includes an amino acid sequence of SEQ ID NO:7 (anti-EGFR1) optionally with one or two or three addition, deletion or substitution.

In some aspects, the first VHH has specificity to an microorganism (e.g., virus or bacterium). Non-limiting examples of microorganism include microorganism surface receptors and endotoxins. Examples of endotoxins include, without limitation, lipopolysaccharide (LPS) and lipooligosaccharide (LOS).

In some aspects, the first VHH includes an amino acid sequence selected from SEQ ID NO: 8-11 (Table 1), or optionally with one or two or three addition, deletion or substitution.

In some aspects, the second VHH (e.g., VHH2 of FIG. 1) has specificity to an immune cell. In one aspect, the immune cell is selected from the group consisting of a T cell, a B cell, a monocyte, a macrophage, a neutrophil, a dendritic cell, a phagocyte, a natural killer cell, an eosinophil, a basophil, and a mast cell.

In one aspect, the second VHH specifically recognizes an antigen selected from the group consisting of CD3, CD16, CD19, CD28 and CD64. In one aspect, the second VHH specifically recognizes CD3 or CD16.

In one aspect, the second VHH has specificity to CD16 or CD3. Representative sequences for this VHH are provided as SEQ ID NO: 2, 3, 4, 5, 12 and 13 (Table 1), or optionally with one or two or three addition, deletion or substitution.

Any of the antibodies or polypeptides described above may further include additional polypeptides, e.g., a signal peptide to direct secretion of the encoded polypeptide, antibody constant regions as described herein, or other heterologous polypeptides as described herein.

It will also be understood by one of ordinary skill in the art that antibodies as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, 95%, 98%, or 99% identical to the starting sequence.

Furthermore, nucleotide or amino acid substitutions, deletions, or insertions leading to conservative substitutions or changes at “non-essential” amino acid regions may be made. For example, a polypeptide or amino acid sequence derived from a designated protein may be identical to the starting sequence except for one or more individual amino acid substitutions, insertions, or deletions, e.g., one, two, three, four, five, six, seven, eight, nine, ten, fifteen, twenty or more individual amino acid substitutions, insertions, or deletions. In certain embodiments, a polypeptide or amino acid sequence derived from a designated protein has one to five, one to ten, one to fifteen, or one to twenty individual amino acid substitutions, insertions, or deletions relative to the starting sequence.

In certain embodiments, an antigen-binding polypeptide comprises an amino acid sequence or one or more moieties not normally associated with an antibody. Exemplary modifications are described in more detail below. For example, a fragment of the disclosure may comprise a flexible linker sequence, or may be modified to add a functional moiety (e.g., PEG, a drug, a toxin, or a label).

Antibodies, variants, or derivatives thereof of the disclosure include derivatives that are modified, i.e., by the covalent attachment of any type of molecule to the antibody such that covalent attachment does not prevent the antibody from binding to the epitope. For example, but not by way of limitation, the antibodies can be modified, e.g., by glycosylation, acetylation, pegylation, phosphorylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the antibodies may contain one or more non-classical amino acids.

In other embodiments, the antigen-binding polypeptides of the present disclosure may contain conservative amino acid substitutions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

Non-limiting examples of conservative amino acid substitutions are provided in the table below, where a similarity score of 0 or higher indicates conservative substitution between the two amino acids.

| | C | G | P | S | A | T | D | E | N | Q | H | K | R | V | M | I | L | F | Y | W |
|---|---|---|---|---|---|---|---|---|---|---|---|---|---|---|---|---|---|---|---|---|
| W | −8 | −7 | −6 | −2 | −6 | −5 | −7 | −7 | −4 | −5 | −3 | −3 | 2 | −6 | −4 | −5 | −2 | 0 | 0 | 17 |
| Y | 0 | −5 | −5 | −3 | −3 | −3 | −4 | −4 | −2 | −4 | 0 | −4 | −5 | −2 | −2 | −1 | −1 | 7 | 10 | |
| F | −4 | −5 | −5 | −3 | −4 | −3 | −6 | −5 | −4 | −5 | −2 | −5 | −4 | −1 | 0 | 1 | 2 | 9 | | |
| L | −6 | −4 | −3 | −3 | −2 | −2 | −4 | −3 | −3 | −2 | −2 | −3 | −3 | 2 | 4 | 2 | 6 | | | |
| I | −2 | −3 | −2 | −1 | −1 | 0 | −2 | −2 | −2 | −2 | −2 | −2 | −2 | 4 | 2 | 5 | | | | |
| M | −5 | −3 | −2 | −2 | −1 | −1 | −3 | −2 | 0 | −1 | −2 | 0 | 0 | 2 | 6 | | | | | |
| V | −2 | −1 | −1 | −1 | 0 | 0 | −2 | −2 | −2 | −2 | −2 | −2 | −2 | 4 | | | | | | |
| R | −4 | −3 | 0 | 0 | −2 | −1 | −1 | −1 | 0 | 1 | 2 | 3 | 6 | | | | | | | |
| K | −5 | −2 | −1 | 0 | −1 | 0 | 0 | 0 | 1 | 1 | 0 | 5 | | | | | | | | |
| H | −3 | −2 | 0 | −1 | −1 | −1 | 1 | 1 | 2 | 3 | 6 | | | | | | | | | |
| Q | −5 | −1 | 0 | −1 | 0 | −1 | 2 | 2 | 1 | 4 | | | | | | | | | | |
| N | −4 | 0 | −1 | 1 | 0 | 0 | 2 | 1 | 2 | | | | | | | | | | | |
| E | −5 | 0 | −1 | 0 | 0 | 0 | 3 | 4 | | | | | | | | | | | | |
| D | −5 | 1 | −1 | 0 | 0 | 0 | 4 | | | | | | | | | | | | | |
| T | −2 | 0 | 0 | 1 | 1 | 3 | | | | | | | | | | | | | | |
| A | −2 | 1 | 1 | 1 | 2 | | | | | | | | | | | | | | | |
| S | 0 | 1 | 1 | 1 | | | | | | | | | | | | | | | | |
| P | −3 | −1 | 6 | | | | | | | | | | | | | | | | | |
| G | −3 | 5 | | | | | | | | | | | | | | | | | | |
| C | 12 | | | | | | | | | | | | | | | | | | | |

In some embodiments, the antibodies may be conjugated to therapeutic agents, prodrugs, peptides, proteins, enzymes, viruses, lipids, biological response modifiers, pharmaceutical agents, or PEG.

The antibodies may be conjugated or fused to a therapeutic agent, which may include detectable labels such as radioactive labels, an immunomodulator, a hormone, an enzyme, an oligonucleotide, a photoactive therapeutic or diagnostic agent, a cytotoxic agent, which may be a drug or a toxin, an ultrasound enhancing agent, a non-radioactive label, a combination thereof and other such agents known in the art.

The antibodies can be detectably labeled by coupling it to a chemiluminescent compound. The presence of the chemiluminescent-tagged antigen-binding polypeptide is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

### Polynucleotides Encoding the Antibodies and Methods of Preparing the Antibodies

The present disclosure also provides isolated polynucleotides or nucleic acid molecules encoding the bispecific antibodies, variants or derivatives thereof of the disclosure.

The polynucleotides of the present disclosure may encode the entire VHH, variants or derivatives thereof on the same polynucleotide molecule or on separate polynucleotide molecules. Additionally, the polynucleotides of the present disclosure may encode portions of the antibody or VHH, variants or derivatives thereof on the same polynucleotide molecule or on separate polynucleotide molecules.

In certain embodiments, the prepared antibodies will not elicit a deleterious immune response in the animal to be treated, e.g., in a human. In one embodiment, antigen-binding polypeptides, variants, or derivatives thereof of the disclosure are modified to reduce their immunogenicity using art-recognized techniques. For example, antibodies can be humanized, primatized, deimmunized, or chimeric antibodies can be made.

The binding specificity of bispecific antibodies of the present disclosure can be determined by in vitro assays such as immunoprecipitation, radioimmunoassay (RIA) or enzyme-linked immunoabsorbent assay (ELISA).

### Production Systems and Methods

The present disclosure also provides systems and methods for producing the bispecific antibody of the present disclosure. Cells suitable for producing antibodies are well known in the art, including human cells (e.g., CHO cells), mammalian cells and bacterial cells. The use of bacterial cells to produce bispecific antibodies presents significant challenges. Nevertheless, as shown in the examples, when expressed in bacterial cells, the resulting antibody is largely soluble, even when both peptide chains are expressed in the same cell.

Therefore, in one embodiment, provided is a host cell comprising one or more polynucleotides encoding both chains of the disclosed bispecific antibody. In one aspect, a single polynucleotide construct (e.g., plasmid) includes both coding sequences. In another aspects, two separate polynucleotide constructs each encodes one of the polypeptide chains. Also provided, in one embodiment, is a host cell comprising both polypeptide chains of the bispecific antibodies of the present disclosure.

In some aspects, the host cells are human cells. In some aspects, the host cells are mammalian cells. In some aspects, the host cells are yeast cells. In some aspects, the host cells are bacterial cells, including Gram-positive and Gram-negative bacterial cells. Representative bacterial cells include, without limitation, E. coli and S. typhymurium.

Also provided, in some aspects, is a method for preparing a bispecific antibody of the present disclosure. In one aspect, the method entails expressing both peptide chains of the antibody in a host cell and extracting the antibody from cell lysis. Further, provided are bispecific antibodies obtained from such methods.

### Treatment and Diagnostic Methods

As described herein, the bispecific antibodies, variants or derivatives of the present disclosure may be used in certain treatments and diagnostic methods associated with cancer or an infectious disease.

The present disclosure is further directed to antibody-based therapies which involve administering the bispecific antibodies of the disclosure to a patient such as an animal, a mammal, and a human for treating one or more of the disorders or conditions described herein. Therapeutic compositions of the disclosure include, but are not limited to, antibodies of the disclosure (including variants and derivatives thereof as described herein) and nucleic acids or polynucleotides encoding antibodies of the disclosure (including variants and derivatives thereof as described herein).

The antibodies of the disclosure can also be used to treat, inhibit or prevent diseases, disorders or conditions including malignant diseases, disorders, or conditions associated with such diseases or disorder such as diseases associated with increased cell survival, or the inhibition of apoptosis, for example cancers (such as follicular lymphomas, carcinomas with p53 mutations, and hormone-dependent tumors, including, but not limited to colon cancer, cardiac tumors, pancreatic cancer, melanoma, retinoblastoma, glioblastoma, lung cancer, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, breast cancer, prostate cancer, Kaposi's sarcoma and ovarian cancer); autoimmune disorders (such as, multiple sclerosis, Sjogren's syndrome, Grave's disease, Hashimoto's thyroiditis, autoimmune diabetes, biliary cirrhosis, Behcet's disease, Crohn's disease, polymyositis, systemic lupus erythematosus and immune-related glomerulonephritis, autoimmune gastritis, autoimmune thrombocytopenic purpura, and rheumatoid arthritis) and viral infections (such as herpes viruses, pox viruses and adenoviruses), inflammation, graft vs. host disease (acute and/or chronic), acute graft rejection, and chronic graft rejection. Antigen binding polypeptides, variants or derivatives thereof of the present disclosure are used to inhibit growth, progression, and/or metastasis of cancers, in particular those listed above or in the paragraph that follows.

Additional diseases or conditions associated with increased cell survival, that may be treated, prevented, diagnosed and/or prognosed with the antibodies or variants, or derivatives thereof of the disclosure include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

The antibodies of the present disclosure can also be used to treat an infectious disease caused by a microorganism, or kill a microorganism, by targeting the microorganism and an immune cell to effect elimination of the microorganism. In one aspect, the microorganism is a virus including RNA and DNA viruses, a Gram positive bacterium, a Gram negative bacterium, a protozoa or a fungus.

A specific dosage and treatment regimen for any particular patient will depend upon a variety of factors, including the particular antigen-binding polypeptide, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compositions used, the severity of the disease, and the desired effect. The amount used can be determined by pharmacological and pharmacokinetic principles well known in the art.

Methods of administration of the bispecific antibodies, variants or include but are not limited to intradermal, intramuscular, intraperitoneal, intravenous, subcutaneous, intranasal, epidural, and oral routes. The antigen-binding polypeptides or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Thus, pharmaceutical compositions containing the antigen-binding polypeptides of the disclosure may be administered orally, rectally, parenterally, intracistemally, intravaginally, intraperitoneally, topically (as by powders, ointments, drops or transdermal patch), bucally, or as an oral or nasal spray.

The term “parenteral” as used herein refers to modes of administration which include intravenous, intramuscular, intraperitoneal, intrasternal, subcutaneous and intra-articular injection and infusion.

Administration can be systemic or local. In addition, it may be desirable to introduce the antibodies of the disclosure into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

It may be desirable to administer the bispecific antibodies or compositions of the disclosure locally to the area in need of treatment; this may be achieved by, for example, and not by way of limitation, local infusion during surgery, topical application, e.g., in conjunction, with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the disclosure, care must be taken to use materials to which the protein does not absorb.

The amount of the antibodies of the disclosure which will be effective in the treatment, inhibition and prevention of an inflammatory, immune or malignant disease, disorder or condition can be determined by standard clinical techniques. In addition, in vitro assays may optionally be employed to help identify optimal dosage ranges. The precise dose to be employed in the formulation will also depend on the route of administration, and the seriousness of the disease, disorder or condition, and should be decided according to the judgment of the practitioner and each patient's circumstances. Effective doses may be extrapolated from dose-response curves derived from in vitro or animal model test systems.

As a general proposition, the dosage administered to a patient of the antigen-binding polypeptides of the present disclosure is typically 0.1 mg/kg to 100 mg/kg of the patient's body weight, between 0.1 mg/kg and 20 mg/kg of the patient's body weight, or 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the disclosure may be reduced by enhancing uptake and tissue penetration (e.g., into the brain) of the antibodies by modifications such as, for example, lipidation.

The methods for treating an infectious or malignant disease, condition or disorder comprising administration of an antibody, variant, or derivative thereof of the disclosure are typically tested in vitro, and then in vivo in an acceptable animal model, for the desired therapeutic or prophylactic activity, prior to use in humans. Suitable animal models, including transgenic animals, are well known to those of ordinary skill in the art. For example, in vitro assays to demonstrate the therapeutic utility of antigen-binding polypeptide described herein include the effect of an antigen-binding polypeptide on a cell line or a patient tissue sample. The effect of the antigen-binding polypeptide on the cell line and/or tissue sample can be determined utilizing techniques known to those of skill in the art, such as the assays disclosed elsewhere herein. In accordance with the disclosure, in vitro assays which can be used to determine whether administration of a specific antigen-binding polypeptide is indicated, include in vitro cell culture assays in which a patient tissue sample is grown in culture, and exposed to or otherwise administered an antibody, and the effect of such an antibody upon the tissue sample is observed.

In a further embodiment, the compositions of the disclosure are administered in combination with an antineoplastic agent, an antiviral agent, antibacterial or antibiotic agent or antifungal agents. Any of these agents known in the art may be administered in the compositions of the current disclosure.

In another embodiment, compositions of the disclosure are administered in combination with a chemotherapeutic agent. Chemotherapeutic agents that may be administered with the compositions of the disclosure include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide).

In an additional embodiment, the compositions of the disclosure are administered in combination with cytokines. Cytokines that may be administered with the compositions of the disclosure include, but are not limited to, IL-2, IL-3, IL-4, IL-5, IL-6, IL-7, IL-10, IL-12, IL-13, IL-15, anti-CD40, CD40L, and TNF-α.

In additional embodiments, the compositions of the disclosure are administered in combination with other therapeutic or prophylactic regimens, such as, for example, radiation therapy.

### Compositions

The present disclosure also provides pharmaceutical compositions. Such compositions comprise an effective amount of an antibody, and an acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, preferably in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### Experimental Examples

### Example 1

### Expression, Purification and Characterization of an Anti-CEA-Fc:Anti-CD16-Fc Antibody

This example tests the expression and purification of an anti-CEA-Fc:anti-CD16-Fc antibody. The antibody has two chains, one with an anti-CEA VHH fragment (SEQ ID NO:1) connected to a Fc fragment (SEQ ID NO:14) and a His6 tag, and another with an anti-CD16 VHH fragment (SEQ ID NO: 12) connected to a Fc fragment (SEQ ID NO:15) and a Flag tag. These two Fc fragments form a knob-in-the-hole pairing.

TABLE 2
Protein Sequences of the Fc Fragments

1. First Fc fragment (Hedge-CH2-CH3) - with T366W modification to
form a knob (SEQ ID NO: 14)
DKTHTCPPCP APELLGGPSV FLFPPKPKDT LMISRTPEVT CVVVDVSHED PEVKFNWYVD
GVEVHNAKTK PREEQYNSTY RVVSVLTVLH QDWLNGKEYK CKVSNKALPA PIEKTISKAK
GQPREPQVYT LPPSRDELTK NQVSLWCLVK GFYPSDIAVE WESNGQPENN YKTTPPVLDS
DGSFFLYSKL TVDKSRWQQG NVFSCSVMHE ALHNHYTQKS LSLSPGK
2. Second Fc fragment (Hedge-CH2-CH3) - with T366S/L368A/Y407V
modifications to form a hole (SEQ ID NO: 15)
DKTHTCPPCP APELLGGPSV FLFPPKPKDT LMISRTPEVT CVVVDVSHED PEVKFNWYVD
GVEVHNAKTK PREEQYNSTY RVVSVLTVLH QDWLNGKEYK CKVSNKALPA PIEKTISKAK
GQPREPQVYT LPPSRDELTK NQVSLSCAVK GFYPSDIAVE WESNGQPENN YKTTPPVLDS
DGSFFLVSKL TVDKSRWQQG NVFSCSVMHE ALHNHYTQKS LSLSPGK

As shown in this example, about 20% of the produced antibody was soluble, which is unexpected as bispecific antibodies produced from a single cell are typically not soluble.

### Materials and Methods

Culture A medium: TB medium supplemented with 2% Glucose, 0.4% Glycerol.

Culture B medium: TB medium supplemented with 0.4% Glycerol, 0.5 mM IPTG.

Lysis buffer 25 mM Tris-HCl, pH 7; 250 mM NaCl; 0.1% Triton-X-100.

Terrific Broth: Deionized H₂O, to 900 mL; Tryptone, 12 g; Yeast extract, 24 g; Glycerol, 4 mL. Shake until the solutes have dissolved and then sterilize by autoclaving for 20 min at 15 psi (1.05 kg/cm²) on liquid cycle. Allow the solution to cool to 60° C. or less, and then add 100 mL of a sterile solution of 0.17 M KH₂PO₄, 0.72 M K₂HPO₄. (This solution was made by dissolving 2.31 g of KH₂PO₄ and 12.54 g of K₂HPO₄ in 90 mL of H₂O. After the salts had dissolved, adjusted the volume of the solution to 100 mL with H₂O and sterilize by autoclaving for 20 min at 15 psi [1.05 kg/cm²] on liquid cycle.)

LB medium: add the following to 800 ml H₂O: 10 g Bacto-tryptone, 5 g yeast extract, 10 g NaCl, adjust pH to 7.5 with NaOH, adjust volume to 1 L with dH₂O, sterilize by autoclaving.

Reagent and Materials for protein purification: binding buffer: 10 mM Tris-HCl; 150 mM NaCl, pH 7.5; elution buffer: 0.1M Glycine, pH 2.7, neutral buffer: 1M Tris pH 9.0; 20% Ethanol.

Transformation

Competent BL21(DE3) E. coli cells were taken out of −80° C. and thaw on ice. About 50 μl competent cells were pipetted to a 1.5 mL pre-chilled tube. One μL DNA construct encoding both antibody chains (concentration of DNA: is about 100 ng/μL) was added to the competent cells, swirled gently and was incubated on ice for 30 min.

Each tube then received a heat shock each, being placed into a 42° C. water bath for 45 s. The tubes were placed back on ice for 2-3 min. 450 μL media was added to each tube, which was then incubated at 37° C., 100 rpm for 1 h.

Fifty μL of the resulting cells were spread on LB plates (Ampencilin, 100 ug/ml, Kanamycin, 50 μg/ml. 37° C., and grown for 12-16 h.

Expression

A single colony from plate was picked to 2 mL TB+antibiotic (Amp+; Kan+; or both Amp+ and Kan+). Two mL cell medium was transferred to 100 mL Culture A medium+antibiotic, 30, 220 rpm to OD₆₀₀˜1. Cells were collected by centrifugation and supernatant was discarded. The cells were resuspended in 2 of 100 ml Culture B medium+antibiotic and grown separately at 16 and 25 for 16 hrs.

One ml cell culture of each time-point was transferred to eppendorf tubes and was centrifuged at 13,000 rpm, 2 min, supernatant discarded. Each tube was added 500 μl PBS (pH7.4) and the resulting pellet was resuspended, and centrifuged: 4, 12000 rpm, 30 min. The supernatant was transferred to another cold fresh tube, with 100 μl lysis buffer to resuspend the pellet.

Coomassie brilliant blue staining was conducted on the antibodies after run on 10 ul SDS-PAGE (15% separating gel). Destainning was done with H₂O. Another 10 ul was used for western blot using either anti-His6 or anti-Flag antibodies. For the rest of culture, the cells were collected by centrifugation at 4000 rpm, 4° C., 20 mins. The cells were resuspended in 10 ml lysis buffer. The samples were frozen on dry ice and stored at −80° C.

Purification Procedure

Collection tubes were prepared by adding 0.1 ml of 1M Tris pH 9.0 per ml of each fraction to be collected. The tubes were centrifuged at 12000 rpm, 4° C. for 30 min or filtered. The samples were dialyzed in 10 mM Tris-HCl, 150 mM NaCl, pH 7.5 for 2 hours at 4° C.

Columns were washed with 5 bed volumes of 10 mM Tris-HCl, 150 mM NaCl, pH 7.5. The samples were applied onto the column, and incubated at 4° C. for 2 hours. The columns were washed with 10 bed volumes of 10 mM Tris-HCl, 150 mM NaCl, pH 7.5, for four times to remove contaminant proteins.

The antibody was eluted with 5 bed volumes of 0.1 M Glycine. Fractions containing the antibody were collected into tubes containing 1M Tris. The samples were dialyzed against 3 at least 100 times the sample volume. Concentrations of the antibody were determined, and then stored as aliquots at −20° C.

Western Blot to Detect Bispecific Antibodies

Twenty μl of the sample was transferred to a 1 mL tube; 5× loading buffer (5 μL) was added. The sample was then boiled for 5 min. The protein was loaded to 8% SDS-PAGE, and transferred to PVDF membrane. The sample was incubated with 5% fat-free milk powder in TBST for 1 hour.

Anti-His-HRP and anti-M2(Flag)-HRP antibodies were used to detect each of the bispecific antibody chains. Washing was carried out with TBST for 3×8 min. Detection solutions was added to the samples before pictures were taken.

Gel Filtration to Determine Whether the Bispecific Antibodies were Aggregates

Materials used: GF column: Superdex 200 3.2/300; molecular weight (Mr) of column: 10 000 to 600,000; Eluent: 25 mM Tris HCl, pH7.5; 300 mM NaCl; Flow rate: 0.05 ml/min, RT.

Concentration of the bispecific antibody was 5.4 mg/ml, in 20 ul. For equilibration, each sample was equilibrated with at least 2 CV of room-temperatured water and then equilibrated with at least 2CV running buffer. Ten ul of protein standard markers (five protein markers of molecular weights: 12.4 to 200 kd were used as standards. Each bispecific antibody sample was loaded and run under the detection of 280 nm.

Cleaning-in-place (CIP) was conducted by washing the column with 1 CV of 0.1 M sodium hydroxide or alternatively 0.5 M acetic acid at a flow rate of 0.02 ml/min. The column was immediately rinsed with 4 CV water followed by at least 4 CV eluent at a flow rate of 0.02 ml/min.

### Results

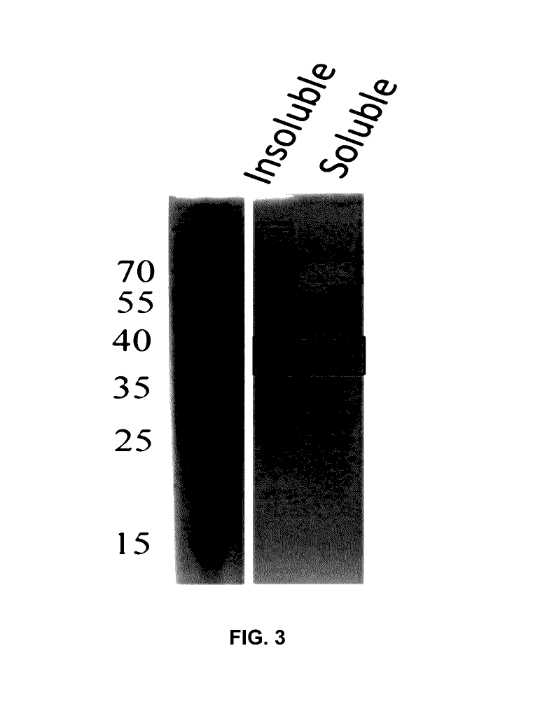

A bispecific antibody with two camel VHHs (anti-CEA and anti-CD16) was expressed in bacteria. FIG. 3 shows Commassiue blue staining of insoluble and soluble fractions of the antibody expressed in the bacterial cells. As shown in the figure, about 20% of the total antibody was soluble.

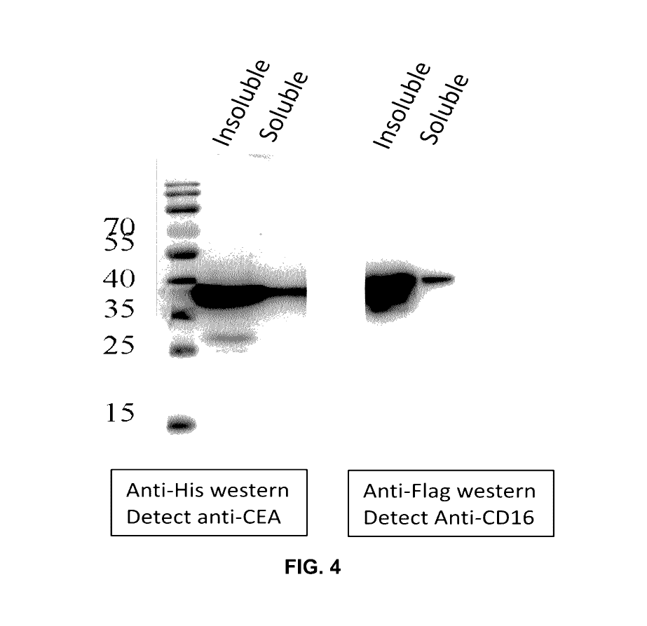

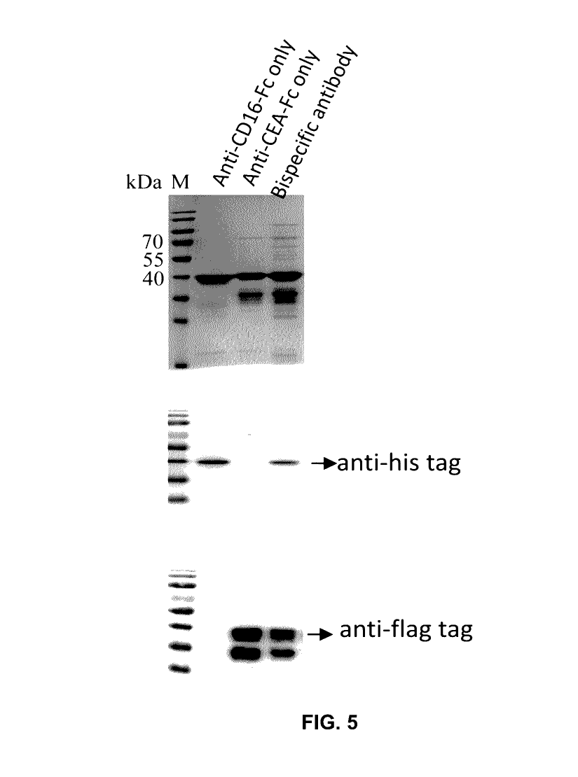

FIG. 4 shows the staining of each of the two antibody chains separately, using antibodies against the His tag and the Flag tag, respectively. As shown in FIG. 4, the two chains were expressed at similar levels. FIG. 5 further shows the staining of each antibody chain alone or when forming the bispecific antibody.

The staining and gel filtration showed that the bispecific antibody has a molecular weight of about 80 KDa, with each chain of about 40 KDa.

The yield of the bispecific antibody was about 1-2 mg per Liter cell culture. Such a yield is greater than other bispecific antibodies produced from two different batches of bacterial cells or mammalian cells to form bispecific antibodies. The purification of the present disclosure is also much easier and needs less time and efforts to control the antibody pairing.

### Example 2

### Preparation of Other Bispecific Antibodies

This example demonstrates the expression and purification of two more bispecific antibodies, an anti-CEA-Fc:anti-CD3-Fc, an anti-Her2-Fc:anti-CD16-Fc and an anti-Her2-Fc:anti-CD3-Fc bispecific antibodies. The production, purification and characterizations methods were the same as used in Example 1. The anti-CEA VHH sequence was SEQ ID NO:1. The anti-CD3 VHH sequence was SEQ ID NO:13. The anti-Her2 VHH sequence was SEQ ID NO:6. The anti-CD16 VHH sequence was SEQ ID NO:12. The Fc fragments had the same sequences as in Example 1.

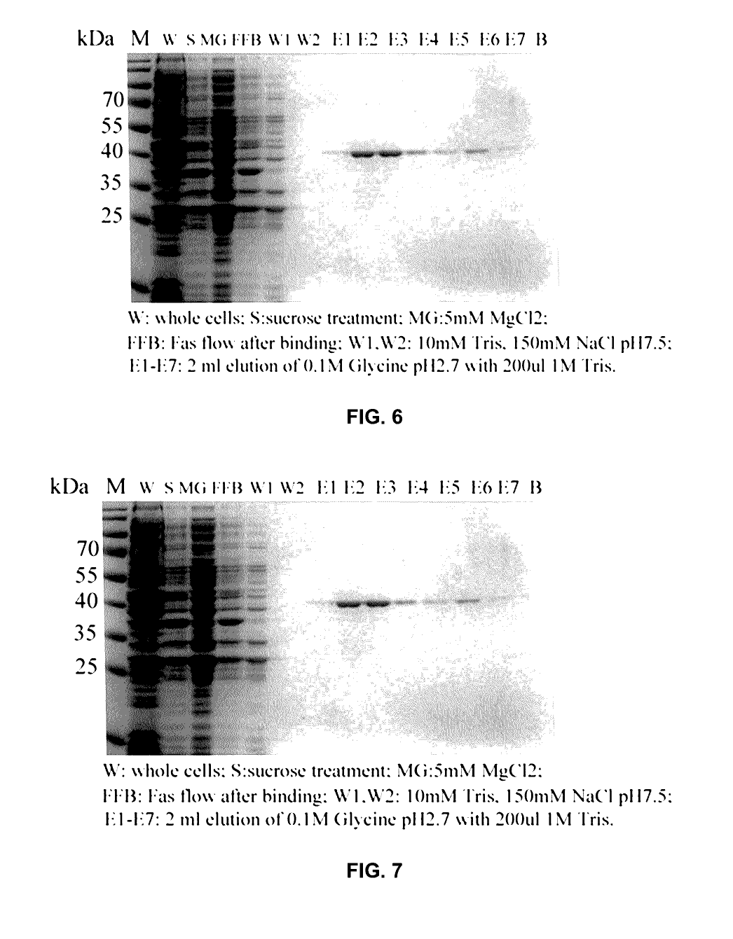

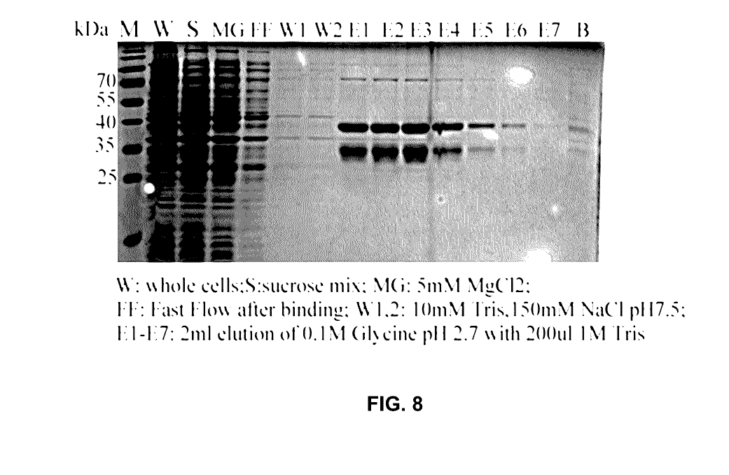

FIG. 6-8 present Western blots images for obtained bispecific antibodies at each stage of purification, for the anti-CEA-Fc:anti-CD3-Fc, the anti-Her2-Fc:anti-CD16-Fc and the anti-Her2-Fc:anti-CD3-Fc bispecific antibodies, respectively. It was also observed that about 20% of the expressed bispecific antibodies were soluble. The yield of each of these antibodies was similar to the one tested in Example 1.

The above two examples, therefore, demonstrate that soluble products of the bispecific antibodies of the present discourse can be efficiently prepared from bacterial cells. Further, such prepared antibodies were stable.

### Example 3

### In Vitro Cell-Based Assays

This example demonstrates that the bispecific antibodies of the present disclosure are effective in targeting tumor cells in an in vitro cytotoxicity assay. The data, therefore, shows that such antibodies can be suitably used clinically to treat cancer.

### Materials and Methods

Cell lines used included HT29 (a CEA positive cell line), SKOV3 (a CEA negative cell line), LS174T (a CEA positive cell line), and human NK cells.

At day 1, the cells were thawed in plated in 10 cm dishes. At day 2, 0.25% trypsin was used to digest every cell lines. Cells were collected and cell numbers counted. The cells were then diluted to 5×10⁴/mL. One hundred μl cell suspension was plated (5000 cells per well) on a 96-well plate. The samples were then incubated for 6 hrs before adding NK cells or T cells.

NK cells or T cells were diluted to 5×10⁵/mL, and 100 μl cells were plated (50,000 cells per well) on the 96-well plates, which contained the tumor cells.

The bispecific antibodies were added to the wells as Table 2 shows, and the sample was incubated for 48 hrs. 20 μl CCK8 reagent was added to every well in the 96-well plates. Data were collected at 1, 2, 3, 4 hrs time points.

TABLE 3
Plate Map

| SKOV3 | | | | HT29 | | | | LS174T | | | |
|---|---|---|---|---|---|---|---|---|---|---|---|
| 1 | 2 | 3 | 4 | 5 | 6 | 7 | 8 | 9 | 10 | 11 | 12 |

| | | | |
|---|---|---|---|
| A | Tumor cells + medium | Tumor cells + medium | Tumor cells + medium |
| B | Tumor cells + NKs/T-cells | Tumor cells + NKs/T-cells | Tumor cells + NKs/T-cells |
| C | Tumor cells + NKs/T-cells + | Tumor cells + NKs/T-cells + | Tumor cells + NKs/T-cells + |
| | BISPECIFIC ANTIBODIES | BISPECIFIC ANTIBODIES | BISPECIFIC ANTIBODIES |
| | 1 μg/ml | 1 μg/ml | 1 μg/ml |
| D | Tumor cells + NKs/T-cells + | Tumor cells + NKs/T-cells + | Tumor cells + NKs/T-cells + |
| | BISPECIFIC ANTIBODIES | BISPECIFIC ANTIBODIES | BISPECIFIC ANTIBODIES |
| | 10 μg/ml | 10 μg/ml | 10 μg/ml |
| E | Tumor cells + BISPECIFIC | Tumor cells + BISPECIFIC | Tumor cells + BISPECIFIC |
| | ANTIBODIES 1 μg/ml | ANTIBODIES 1 μg/ml | ANTIBODIES 1 μg/ml |
| F | Tumor cells + NKs/T-cells + | Tumor cells + NKs/T-cells + | Tumor cells + NKs/T-cells + |
| | BISPECIFIC ANTIBODIES | BISPECIFIC ANTIBODIES | BISPECIFIC ANTIBODIES |
| | 1 μg/ml | 1 μg/ml | 1 μg/ml |
| G | Tumor cells + BISPECIFIC | Tumor cells + BISPECIFIC | Tumor cells + BISPECIFIC |
| | ANTIBODIES 10 μg/ml | ANTIBODIES 10 μg/ml | ANTIBODIES 10 μg/ml |
| H | Tumor cells + NKs/T-cells + | Tumor cells + NKs/T-cells + | Tumor cells + NKs/T-cells + |
| | BISPECIFIC ANTIBODIES | BISPECIFIC ANTIBODIES | BISPECIFIC ANTIBODIES |
| | 10 μg/ml | 10 μg/ml | 10 μg/ml |

### Results

The cytotoxicity assays used monospecific antibodies as controls and included both tumor cells that expressed the targeted tumor antigen and those that did not express the targeted antigen. The bispecific antibodies' abilities to destroy tumor cells were pronounced.

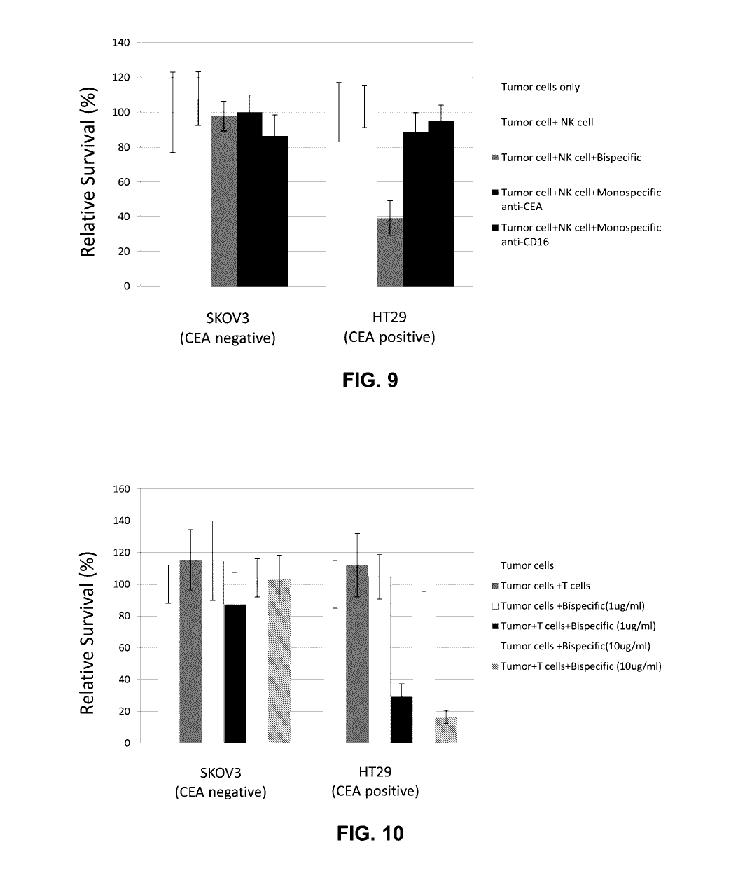

Compared to the monospecific antibodies, the anti-CEA-Fc:anti-CD16-Fc bispecific antibody was about twice as effective at CEA-positive cells (FIG. 9, right panel) in the presence of NK cells. Such a difference was not observed for CEA-positive cells (FIG. 9, left panel).

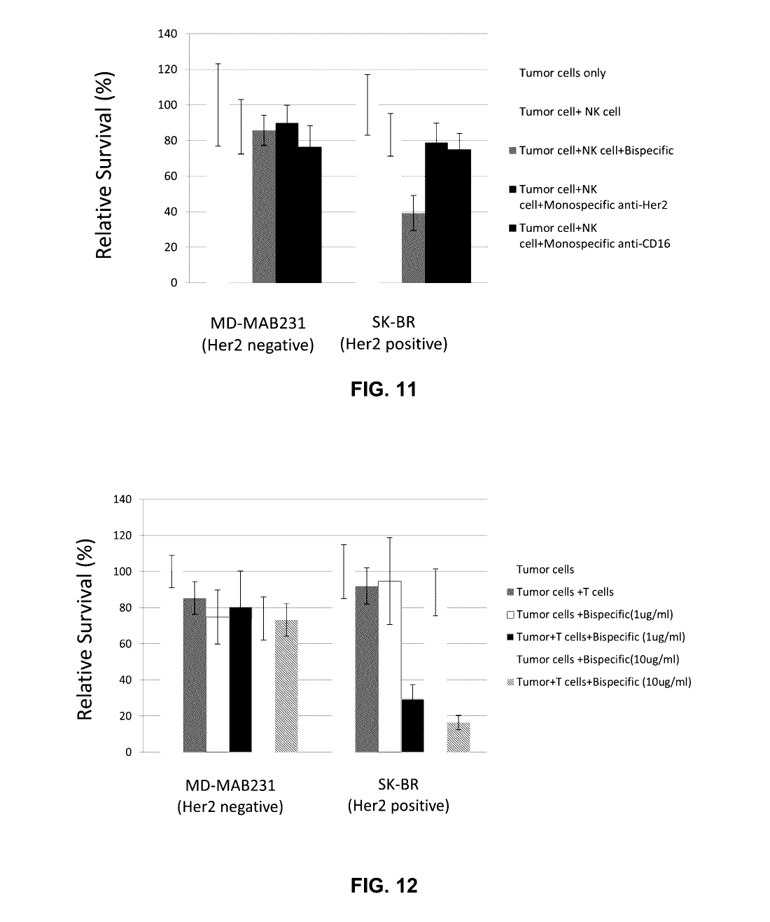

The advantage of the anti-CEA-Fc:anti-CD16-Fc bispecific antibody was even more dramatic over the corresponding monospecific antibodies. Only about 30% of CEA-positive tumor cells (in the presence of T cells) survived when treated with the bispecific antibody, as compared to the monospecific counterpart (FIG. 10). Likewise, the anti-CEA-Fc:anti-CD3-Fc bispecific antibody exhibited greatly improved cytotoxicity at Her2-positive tumor cells in the presence of NK cells (FIG. 11).

FIG. 12 further shows that the cytotoxicity of these bispecific antibodies were dose-dependent (compare the fourth bar and the sixth bar in the right panel) and immune cell-dependent (compare the fourth bar and the fifth bar in the right panel).

To the best knowledge of the inventors, these data show that the potency of these bispecific antibodies is similar to other bispecific antibodies in the literature. Given the much improved stability and production efficiency of these bispecific antibodies, especially the ability to be produced from bacterial cells, these bispecific antibodies show great potential for further development and clinical use for cancer treatment.

It should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

The disclosures illustratively described herein may suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

1. A bivalent bispecific antibody comprising (a) a first polypeptide comprising a first Fc fragment and a first single-domain antigen-binding (VHH) fragment and (b) a second polypeptide comprising a second Fc fragment and a second VHH fragment, wherein the first VHH fragment has specificity to a tumor cell and the second VHH fragment has specificity to an immune cell.

2. The antibody of claim 1, containing no antibody light chains.

3. The antibody of claim 1, wherein the first VHH fragment has specificity to a tumor antigen.

4. The antibody of claim 3, wherein the tumor antigen is selected from the group consisting of CEA, EGFR, Her2, EpCAM, CD20, CD30, CD33, CD47, CD52, CD133, CEA, gpA33, Mucins, TAG-72, CIX, PSMA, folate-binding protein, GD2, GD3, GM2, VEGF, VEGFR, Integrin, αVβ3, α5β1, ERBB2, ERBB3, MET, IGFIR, EPHA3, TRAILR1, TRAILR2, RANKL, FAP and Tenascin.

5. The antibody of claim 3, wherein the tumor antigen is CEA or Her2.

6. The antibody of claim 1, wherein the first VHH fragment comprises the amino acid sequence of SEQ ID NO:1 or 6, or an amino acid having at least about 95% sequence identity thereto.

7. The antibody of claim 1, wherein the second VHH fragment has specificity to a mammalian T cell or a mammalian NK cell.

8. The antibody of claim 7, wherein the second VHH fragment has specificity to an antigen selected from the group consisting of CD3, CD16, CD19, CD28 and CD64.

9. The antibody of claim 7, wherein the antigen is CD16 or CD3.

10. The antibody of claim 7, wherein the second VHH fragment comprises the amino acid sequence of one of SEQ ID NO:2-5 or 12-13, or an amino acid having at least about 95% sequence identity thereto.

11. The antibody of claim 1, wherein the first VHH fragment and/or the second VHH fragment does not contain Val, Gly, Leu, and Trp residues at Kabat positions 37, 44, 45, and 47, respectively.

12. The antibody of claim 1, wherein each of the Fc fragments comprises a CH2 domain and a CH3 domain.

13. The antibody of claim 1, wherein the first Fc fragment and the second Fc fragment each comprises a different amino acid sequence selected from SEQ ID NO:14 or SEQ ID NO:15.

14. A polypeptide comprising the amino acid sequence of SEQ ID NO:13 or having at least 95% sequence identity to SEQ ID NO:13, wherein the polypeptide has binding specificity to a mammalian CD3 protein.

15. The polypeptide of claim 1, wherein the mammalian CD3 protein is a human CD3 protein.

16. A bivalent antibody comprising (a) a first polypeptide comprising a first Fc fragment and a first single-domain antigen-binding (VHH) fragment and (b) a second polypeptide comprising a second Fc fragment and a second VHH fragment.
